(19) United States
(12) Patent Application Publication
(10) Pub. No.: US 2025/0253050 A1
(43) Pub. Date: Aug. 7, 2025

(54) INTERACTIVE TOOL TO IMPROVE RISK PREDICTION AND CLINICAL CARE FOR A DISEASE THAT AFFECTS MULTIPLE ORGANS

(71) Applicant: THE JOHNS HOPKINS UNIVERSITY, Baltimore, MD (US)

(72) Inventors: Ji Soo KIM, Baltimore, MD (US); John SCOTT, Baltimore, MD (US); Laura HUMMERS, Baltimore, MD (US); Scott ZEGER, Baltimore, MD (US); Ami SHAH, Ellicott City, MD (US)

(21) Appl. No.: 18/855,172

(22) PCT Filed: May 4, 2023
(86) PCT No.: PCT/US2023/021009

§ 371 Date: Oct. 8, 2024

Related U.S. Application Data
(60) Provisional application No. 63/339,759, filed on May 9, 2022.

(51) Int. Cl. G16H 50/30; G16H 10/60; G16H 50/70

(52) U.S. Cl. CPC G16H 50/30; G16H 10/60; G16H 50/70

(57) ABSTRACT

A method, a system, and a non-transitory computer-readable medium provides an interactive patient-level data visualization and analysis tool that illustrates a patient's health trajectory across multiple organ systems. Data from an electronic medical record system and one or more research databases are integrated into an analytics platform. A visualization tool plots the patient's health trajectory and overlays data from an entire user-defined disease cohort as a reference group to visualize a disease course of the patient compared to courses of other patients, with a same disease, selected by a user.

### CROSS-REFERENCE TO RELATED APPLICATIONS

This application is the U.S. national phase of PCT Application No. PCT/US2023/021009, filed in the U.S. Receiving Office on May 4, 2023, which claims the benefit of U.S. Provisional Patent Application No. 63/339,759, filed in the U.S. Patent and Trademark Office on May 9, 2022, and entitled “INTERACTIVE TOOL TO IMPROVE RISK PREDICTION AND CLINICAL CARE FOR A DISEASE THAT AFFECTS MULTIPLE ORGANS.” U.S. Provisional Patent Application No. 63/339,759 is hereby incorporated by reference in its entirety.

### STATEMENT REGARDING FEDERALLY SPONSORED RESEARCH

This invention was made with government support under grant nos. AR070254, AR073208 and AR080217 awarded by the National Institutes of Health. The government has certain rights in the invention.

### BACKGROUND OF THE INVENTION

Rare autoimmune diseases such as, for example, scleroderma as well as other chronic, multisystem diseases are complex and heterogeneous diseases with high variability in clinical phenotype, longitudinal trajectory, treatment response and mortality. For example, scleroderma can affect multiple organ systems including skin, peripheral vasculature, heart, lung, kidneys, muscles, and joints. It has been estimated that most systemic sclerosis (scleroderma) complications (cardiac involvement, pulmonary hypertension, clinically significant interstitial lung disease (ILD), renal crisis, myositis, inflammatory arthritis, digital ulcers, cancer) occur in ˜15% of systemic sclerosis patients. While many risk factors have been identified for these complications at the population level, these have not been easily translatable to clinical practice at the patient level to inform targeted screening or early intervention. For example, it is known that African Americans, those with diffuse cutaneous scleroderma or those with anti-topoisomerase 1 antibodies have a higher risk of clinically significant ILD. However, for an individual patient, it remains unknown how this risk is modified by the presence of multiple risk factors, is affected by the individual patient's own pulmonary function trajectory early in the disease, or changes with involvement of other organ systems.

In a clinic, physicians use cognitive skills to integrate information across multiple parameters and organ systems, factoring in a patient's prior health trajectory and baseline risk factors, to make estimates about a patient's health state, risk for complications, and need for high-risk therapies. This process is informed by a physician's prior experiences caring for patients with a similar expression of disease, and therefore is not generalizable across providers—particularly in a rare disease. Aggregating this complex, longitudinal data for clinical use requires a tremendous time investment on the part of a treating provider. It is also challenging to clearly explain this information to patients during a routine clinical visit to facilitate shared decision making. Lastly, because diseases such as scleroderma as well as other chronic, multisystem diseases can be complex and rare, it is often difficult to address questions of importance to patients, such as: what is the current status of my disease; what is my future likely to hold; and how do I compare with other patients who have the same disease?

### SUMMARY OF THE INVENTION

In a first embodiment, a method provides an interactive patient-level data visualization and analysis tool that illustrates a patient's health trajectory across multiple organ systems. According to the method, data tables from an electronic medical record system and one or more research databases are integrated into an analytics platform. A visualization tool plots the patient's health trajectory and overlays data from an entire user-defined disease cohort as a reference group to visualize a disease course of the patient compared to courses of other patients, with a same disease, selected by a user.

In a second embodiment, a system provides an interactive patient-level data visualization and analysis tool that illustrates a patient's health trajectory across multiple organ systems. The system includes a processor and a memory connected with the processor. The memory includes computer-readable instructions for the processor to perform operations. According to the operations, data tables from an electronic medical record system and one or more research databases are integrated into an analytics platform. A visualization tool plots the patient's health trajectory and overlays data from an entire user-defined disease cohort as a reference group to visualize a disease course of the patient compared to courses of other patients, with a same disease, selected by a user.

In a third embodiment, a non-transitory computer-readable medium has instructions stored thereon for a processor to perform operations. According to the operations, data tables from an electronic medical record system and one of more research databases are integrated into an analytics platform. A visualization tool plots a patient's health trajectory and overlays data from an entire user-defined disease cohort as a reference group to visualize a disease course of the patient compared to courses of other patients, with a same disease, selected by a user.

### BRIEF DESCRIPTION OF THE DRAWINGS

FIG. 1 illustrates an example environment in which various embodiments may be implemented.

FIG. 2 shows an example computing system that may implement either a server and/or a user's computing device according to various embodiments.

FIG. 3 shows multiple data sources, including an electronic medical record (EMR), being ingested into an analytics platform, where data are harmonized according to various embodiments.

FIG. 4 shows a multistep process for updating real-time patient data according to embodiments.

FIG. 5 illustrates a high level view of processing that may be performed according to embodiments.

FIG. 6 shows example output of a visualization and analytics tool, according to various embodiments, illustrating a patient's longitudinal cardiac and medication data.

FIG. 7a shows lung trajectories of a patient and 10^{th}, 50^{th}, and 90^{th} percentile reference lines on an overall scleroderma population according to various embodiments.

FIG. 7b shows lung trajectories of a same patient as shown in FIG. 5a and 10^{th}, 50^{th}, and 90^{th} percentile reference lines of a selected subpopulation.

FIGS. 8a-8c show an example display of a visualization and analytics tool in which users may select variables of interest to view.

FIG. 9 shows an example visualization and analytics tool interface that illustrates an individual patient's health trajectory in several parameters over time with an estimated risk of crossing high risk thresholds in these parameters within a next 6 month time period.

### DETAILED DESCRIPTION OF THE INVENTION

To address the above mentioned challenges, a tool was designed that communicates a patient's longitudinal data across multiple organ systems and illustrates the patient's health vector relative to other patients with a same disease. Embodiments of the tool may include interactive filters that enable a healthcare provider to compare an individual patient to a subgroup of patients who share relevant clinical and biological characteristics. A prototype was implemented in a web based application programming interface that can be viewed within different electronic medical record (EMR) systems to bring the tool within clinicians' workflow and enable future dissemination. Embodiments of the tool may have embedded therein computed personalized risk estimates for major disease complications, harnessing knowledge from a patient's prior health trajectory in multiple organ systems and known outcomes from patients with similar subgroup characteristics. While examples in this communication focus on scleroderma, the methods described herein have broad applicability across complex, multisystem diseases and health systems.

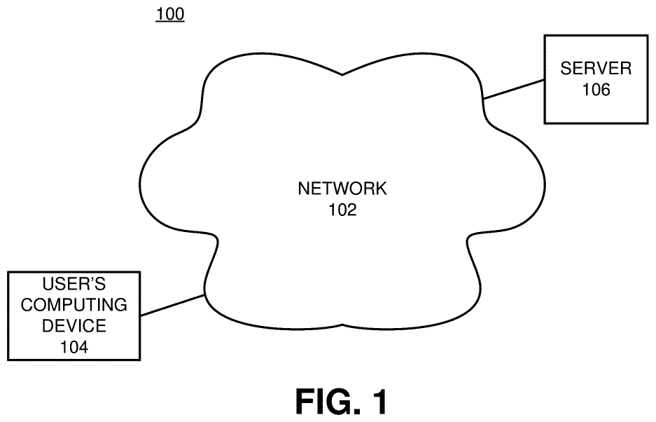

FIG. 1 illustrates an example environment 100 in which various embodiments may be implemented. Environment 100 may include a network 102, which may be a wired or wireless network or combination thereof. In some embodiments, network 102 may include multiple networks such as, for example, the Internet.

Network 102 may be implemented by any number of any suitable communications media (e.g., wide area network (WAN), local area network (LAN), Internet, Intranet, etc.) or a combination of any of the suitable communications media. Network 102 may further include wired and/or wireless networks.

User's computing device 104 may include a desktop computer, a laptop computer, a smartphone, a tablet computer, or other type of computing device and may be connected to network 102 via a wired or wireless connection.

Server 106 may include a single computer or may include multiple computers configured as a server farm. The one or more computers of server 106 may include a mainframe computer, a desktop computer, or other types of computers. Server 106 may be connected to network 102 via a wired or a wireless connection. In some embodiments, server 106 may reside in a cloud.

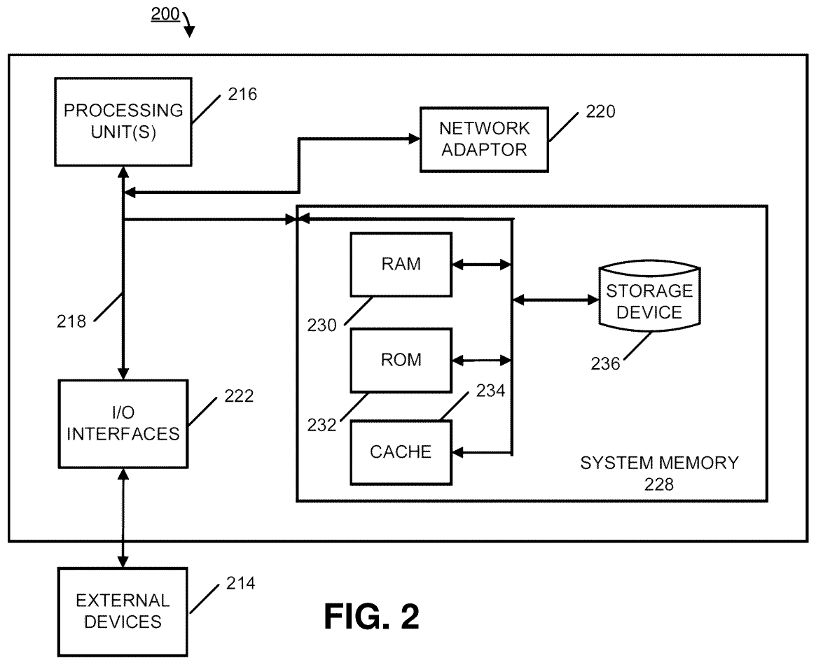

FIG. 2 illustrates an example computing system 200 that may implement any of server 106 and/or user's computing device 104. Computing system 200 is shown in a form of a general-purpose computing device. Components of computing system 200 may include, but are not limited to, one or more processing units 216, a system memory 228, and a bus 218 that couples various system components including system memory 228 to one or more processing units 216.

Bus 218 represents any one or more of several bus structure types, including a memory bus or memory controller, a peripheral bus, an accelerated graphics port, and a processor or local bus using any of a variety of bus architectures. Such architectures may include, but not be limited to, Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computing system 200 may include various non-transitory computer system readable media, which may be any available non-transitory media accessible by computing system 200. The computer system readable media may include volatile and non-volatile non-transitory media as well as removable and non-removable non-transitory media.

System memory 228 may include non-transitory volatile memory, such as random access memory (RAM) 230 and cache memory 234. System memory 228 also may include non-transitory non-volatile memory including, but not limited to, read-only memory (ROM) 232 and storage system 236. Storage system 236 may be provided for reading from and writing to a nonremovable, non-volatile magnetic medium, which may include a hard drive or a Secure Digital (SD) card. In addition, a magnetic disk drive, not shown, may be provided for reading from and writing to a removable, non-volatile magnetic disk such as, for example, a floppy disk, and an optical disk drive for reading from or writing to a removable non-volatile optical disk such as a CD-ROM, DVD-ROM or other optical media. Each memory device may be connected to bus 218 by at least one data media interface. System memory 228 further may include instructions for processing unit(s) 216 to configure computing system 200 to perform functions of embodiments of the invention. For example, system memory 228 also may include, but not be limited to, processor instructions for an operating system, at least one application program, other program modules, program data, and an implementation of a networking environment.

Computing system 200 may communicate with one or more external devices 214 including, but not limited to, one or more displays, a keyboard, a pointing device, a speaker, at least one device that enables a user to interact with computing system 200, and any devices including, but not limited to, a network card, a modem, etc. that enable computing system 200 to communicate with one or more other computing devices. The communication can occur via Input/Output (I/O) interfaces 222. Computing system 200 can communicate with one or more networks including, but not limited to, a local area network (LAN), a general wide area network (WAN), a packet-switched data network (PSDN) and/or a public network such as, for example, the Internet, via network adapter 220. As depicted, network adapter 220 communicates with the other components of computer system 200 via bus 218.

It should be understood that, although not shown, other hardware and/or software components could be used in conjunction with computer system 200. Examples, include, but are not limited to, microcode, device drivers, redundant processing units, external disk drive arrays, RAID systems, tape drives, and data archival storage systems, etc.

The John Hopkins Scleroderma Center Research Registry Center has a dynamic entry, prospective longitudinal cohort that includes all consenting patients who meet 1980 or 2013 American College of Rheumatology classification criteria for scleroderma, have at least 3 of 5 features of the CREST (calcinosis, Raynaud's phenomenon, esophageal dysmotility, sclerodactyly, telangiectasias), or have definite Raynaud's phenomenon, abnormal nailfold capillaries and a scleroderma-specific autoantibody. Data from consenting registry participants had been ingested into an analytics platform known as the Johns Hopkins University Precision Medicine Analytics platform (PMAP). Although, in other embodiments, an analytics platform other than PMAP could be used.

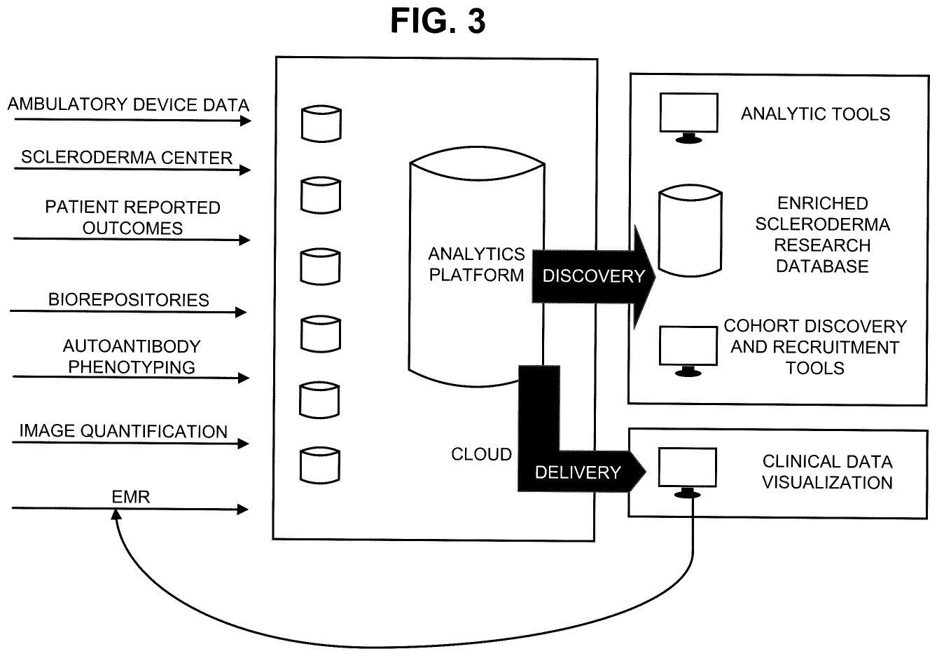

FIG. 3 shows multiple data sources, including an EMR, being ingested into an analytics platform such as the PMAP, where data are harmonized. The multiple sources may include, but not be limited to, ambulatory device data (i.e., smartwatches, at home spirometers, etc.), scleroderma center data, patient reported outcomes, biorepositories, autoantibody phenotyping, image quantification, and EMR data. Enriched data may be used for research studies and data analysis in a Discovery Platform of the analytics platform. The data are also brought back into a context of clinical care through the development of applications in a Delivery Platform of the analytics platform. A clinical data visualization tool, Patient Insight, is one such delivery application developed for the Scleroderma Center context.

In various embodiments, the data from the multiple sources may be ingested into the analytics platform in real-time using FHIR® technology (Fast Healthcare Interoperability Resources) (FHIR is a registered trademark of Health Level Seven International, Inc., DBA Health Level Seven International, a New Jersey Corporation).

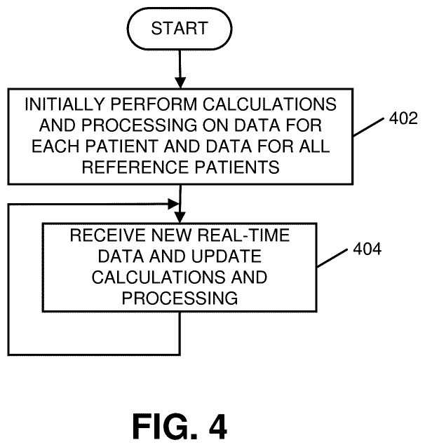

Due to the quantity of the data and complex calculations to be performed on the data, processing of the data could take days or weeks to process if done all at once. In the various embodiments, the calculations and processing may be performed in multiple steps as shown in FIG. 4. For example, the calculations and processing of the data received before a current doctor visit may initially be performed on each patent's data individually and on all reference patients' data collectively (act 402). After new real-time data is received, the calculations and processing for a corresponding patient and all reference patients may be updated (act 404). Act 404 may be performed each time new real-time data is received.

In a current practice of medicine, a clinician has access to historical and current data only about a patient at hand. The historical and current data may come from multiple different sources, making it difficult to aggregate or visualize trends for an individual patient. Further, data from other similar patients are not readily available to inform decision-making. Clinicians therefore make qualitative judgements about the patient's status, health trajectory, and likely benefits of different treatments, not fully informed by either the patient's own data or experiences for similar patients. To address these limitations, an analytics platform, including but not limited to PMAP, may be used to harmonize internal and external streams of data, and uniquely, bring patient-level data and population-level data back into a context of clinical care.

In order to improve patient care, in various embodiments, a data science tool such as a visualization and analysis (VA) tool is embedded within a clinical workflow to guide physician interactions with patients. The VA tool was initially developed in prototype form as an R Shiny application. However, other embodiments of the VA tool may be developed using another web application package or statistical package that builds interactive web applications. Expert clinicians selected key clinical information to be displayed and reviewed and approved preliminary versions. R Shiny App features may be implemented into a longitudinal viewer including a web-based application programming interface that could be viewed within an EMR system including but not limited to Epic. This step met two objectives: (i) to generate a version of the tool that physicians can use directly to test its value in clinical care and (ii) to enable future dissemination of the tool across health systems and EMR platforms. The web-based version of the VA tool may be updated outside of the EMR allowing for rapid iterations and improvements.

The VA tool illustrates a patient's aggregate clinical phenotype in a snapshot view, including cutaneous subtype, cumulative disease manifestations, disease onset dates and autoantibody status. In some embodiments, any history of the following features may be listed as disease manifestations: interstitial lung disease (ILD), pulmonary arterial hypertension, renal crisis, tendon friction rubs, synovitis, myopathy, calcinosis, and other components of the 2013 American College of Rheumatology classification criteria for systemic sclerosis. Comorbid conditions such as peripheral artery disease, coronary artery disease, atherosclerotic cerebrovascular disease, hypertension, and cancer may also be captured.

Longitudinal data may be illustrated across multiple organ systems including but not limited to: 1) cardiac (left ventricular ejection fraction (LVEF), right ventricular systolic pressure (RVSP), and right heart catheterization data), 2) pulmonary (percent predicted forced vital capacity—pFVC and diffusing capacity—pDLCO), 3) cutaneous (modified Rodnan skin score—mRSS), 4) gastrointestinal (Medsger GI severity scores and body mass index), 5) peripheral vasculature (Medsger Raynaud's scores capturing damage including digital pits, ulcerations and gangrene, and 6) muscle (proximal muscle strength on a 0-5 scale). Additionally, patient reported outcome measures have been incorporated, such as, for example, the Scleroderma Health Assessment Questionnaire (HAQ) Disability Index (DI) scores, and laboratory data over time. Longitudinal immunosuppressive medication exposure data may also be shown to assess whether drug exposure alters trajectory in these parameters.

One goal was to enable quick visualization of critical events over time and how they may relate across organ systems. Critical events were defined by either (i) having longitudinal observations exceed or fall below pre-specified thresholds or (ii) having a discrete event occur at a particular date (such as renal crisis or cancer diagnosis). In some embodiments, events were defined as follows: clinically significant ILD (pFVC<70% of predicted), severe ILD (pFVC<60% of predicted), cardiomyopathy (LVEF<50%), pulmonary hypertension (PH) (RVSP≥45 mmHg or mean pulmonary arterial pressure (PAP)≥20 mmHg or ≥25 mmHg for patients with right heart catheterization (RHC) data), severe GI dysmotility (requiring total parenteral nutrition (TPN) or a feeding tube), myopathy (proximal muscle weakness with creatine kinase (CK) elevation, myopathic electromyogram, muscle edema on magnetic resonance imaging, or abnormal muscle biopsy), renal crisis, or cancer diagnosis. These events may be plotted on a single time scale starting from disease onset.

The VA tool may incorporate multiple percentile values such as, for example, a 10^{th} percentile value, a 50^{th} percentile value, and a 90^{th} percentile value for an entire scleroderma cohort or other disease cohort as a reference group. By plotting individuals' health trajectories on top of these reference lines, a patient's disease course can be visualized and compared to others. Moreover, filters may be programmed to compare a patient's health trajectory to a user-specified subgroup based on demographic, clinical and biological characteristics. This allows clinicians to monitor a patient's disease course relative to a group of similar patients based upon known risk factors, such as, for example, age at scleroderma (or other disease) onset, race, sex, cutaneous subtype, and autoantibody status.

A patient's true health state is an unobserved (“latent”) construct reflected in their longitudinal measurements and occurrences of sentinel events. The disease status in multiple organs is measured longitudinally at irregular times and with error. Each patient's disease state and rate of progression is optimally estimated at any given moment and then communicated by integrating them within the VA tool. By fully utilizing information in multiple longitudinal markers, the precision of estimates of patient-specific and population trajectories are maximized.

In some embodiments, the latent health state is modeled since individuals' disease onset using cardiopulmonary and cutaneous parameters (pFVC, pDLCO, LVEF, RVSP and mRSS). As is the clinical tradition in scleroderma, disease onset may be defined by an earlier of onset of Raynaud's phenomenon and first non-Raynaud's symptom.

Patient trajectories are optimally estimated using a Bayesian multivariate linear mixed effects model (MLMM). For each outcome measure, a set of regression predictors (covariates) are selected for fixed effects. Here, age of scleroderma (or other disease) onset, race, biological sex, cutaneous subtype, and autoantibody status (anti-centromere (ACA), anti-topoisomerase 1 (Scl-70), and anti-RNA polymerase III (RNAPol)) may be used. In other embodiments, additional or other regression predictors may be used. To model changes in patients' disease trajectories in time since onset, a smooth function of time may be included using natural splines. For patient specific random effects, a random slope and intercept and two linear splines may be fitted at 3 and 10 years from a current visit to make predictions of current and future health states rely on more recent data. Using model estimates, disease trajectories may be calculated and displayed for each patient for the clinically selected measures such as, for example, pFVC, RVSP, and EF. These were chosen because they are important surrogates for key outcome measures, including ILD, pulmonary hypertension and cardiomyopathy. In other embodiments, additional or other outcome measures may be used.

The estimated current level and recent trend of a patient's disease trajectories predict a risk of having extreme values of these parameters in the near future. Observations falling below or rising above a clinically set threshold are useful surrogates for critical events that will likely require immediate medical attention sometimes followed by more invasive and higher risk interventions. For example, LVEF<35% implies severe heart failure and patients are often treated with an implantable cardioverter defibrillator (ICD).

To predict critical events, each individual's health trajectory is projected into a future, then a probability may be calculated regarding whether the each individual will cross the following boundaries: LVEF<50% and LVEF<35% (cardiomyopathy), RVSP≥45 mmHg and RVSP≥50 mmHg (PH), and pFVC≤70% and pFVC≤60% (ILD) in the next 6, 12, 18, and 24 months. The estimation of these event probabilities may use our methodology called Cross-Validated Sequential Prediction (CVSP). A CVSP algorithm sequentially produces a most likely trajectory and a risk of clinical events as additional data points are observed for a patient. The predictions are made without refitting the model to incorporate new observations for a patient using a cross-validation method. CVSP increases in precision as more data are observed for a given patient, and even with no observations for an individual's measure, CVSP yields predictions with considerable precision compared to other methods.

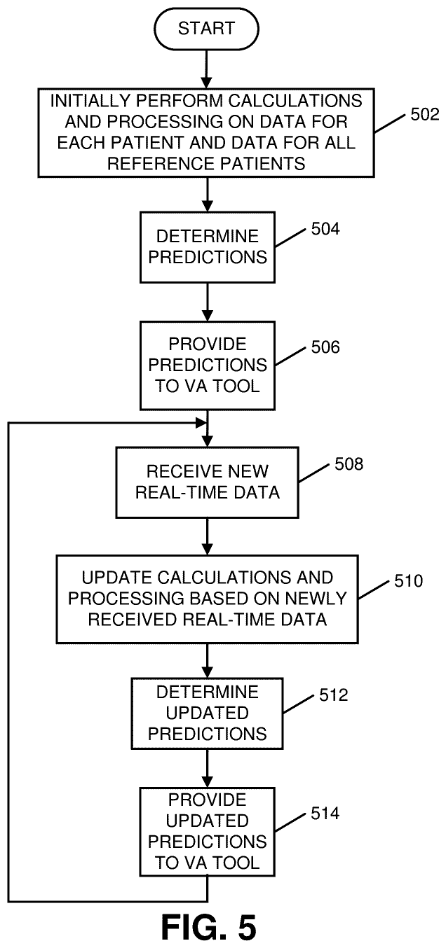

FIG. 5 provides a high level view of the processing performed in an embodiment. The processing may begin by initially performing calculations and processing on data received from multiple sources for each patient and data for all reference patients (act 502). Predictions then may be determined based on the received data (act 504). The determined predictions then may be provided to the VA tool (act 506). New real-time data then may be received (act 508) and the calculations and processing may be updated based on the newly received real-time data (act 510). Updated predictions then may be determined (act 512) and the updated predictions may be provided to the VA tool (act 514). Acts 508-514 may be repeated when new real-time data is received.

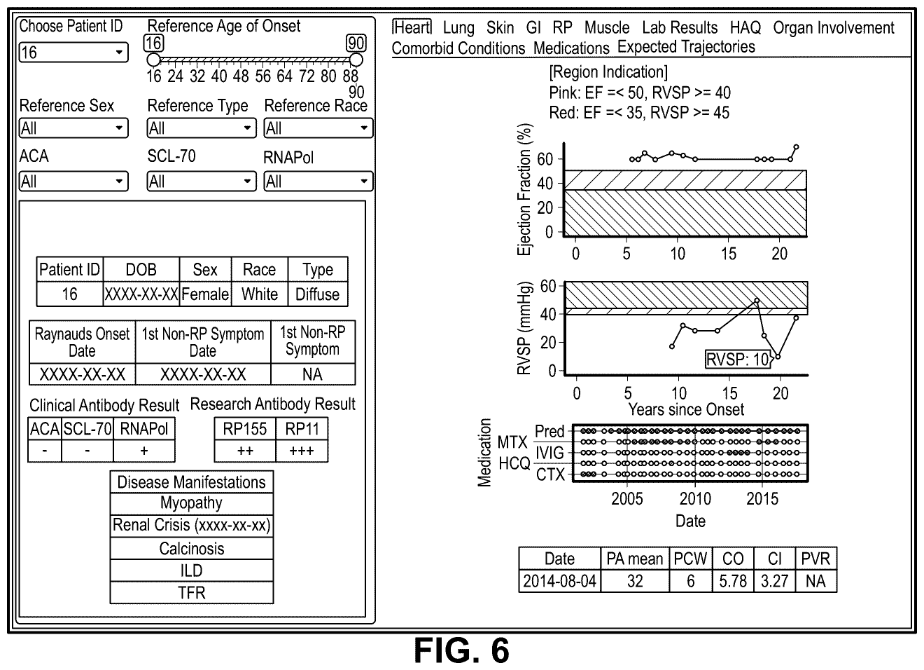

FIG. 6 shows example output of a VA tool illustrating a patient's longitudinal cardiac and medication data. On a left panel, a patient's demographic and clinical disease manifestations are displayed. On a right panel, longitudinal cardiac trajectory is illustrated. Not shown in FIG. 6, a severity of disease state may be indicated by red (severe) and pink (mild) regions for left ventricular ejection fraction (EF) and right ventricular systolic pressure (RVSP). Users can view values for each point and associated medical record information by hovering over the points in the graphs. In a medication plot, colors (not shown in FIG. 6) and connections between the points represent the patterns of medication exposure. Points that are colored black may indicate that the patient is currently on medication, points that are colored green may indicate that the patient is currently not on medication, but was on medication less than 6 months prior to a visit. Points that are colored grey may indicate that the patient is not on medication. The method is informative in that it separates missing medication data (no point plotted) from no exposure (grey), prior exposure only (green), and current exposure (black). Data from right heart catheterizations (RHC) are displayed for patients who had the procedure at a bottom of the tab.

In FIG. 6, the visualization and analytics tool illustrates a patient's aggregate clinical phenotype and longitudinal data in a snapshot view in R Shiny App. Tabs show an individual patient's trajectory across multiple organ systems (cardiac, pulmonary, cutaneous, GI, renal, peripheral vascular, muscle), HAQ-DI, critical events, comorbid conditions, or expected trajectories. The selected tab in FIG. 6 shows the patient's cardiac data over time. Information that is displayed in each tab in the visualization and analytics tool may be presented in a single page view that users can scroll through.

Below the longitudinal display of cardiac measures, longitudinal immunosuppressive medication exposure for the patient is shown. Medications are also displayed in tabs for other organ systems so that a clinician can view a patient's organ-specific trajectory in the context of relevant drug exposures.

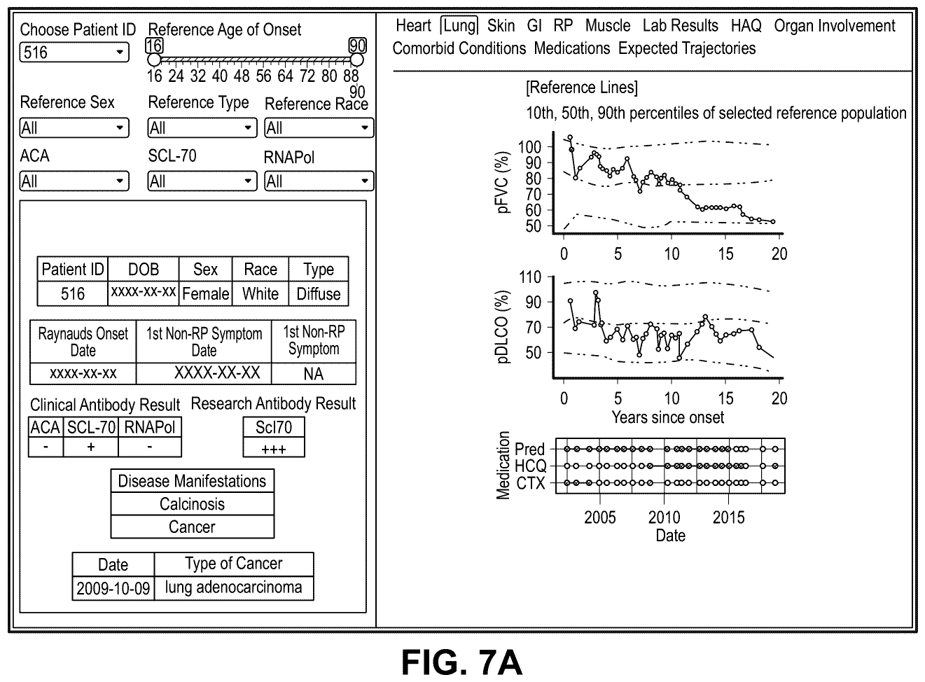

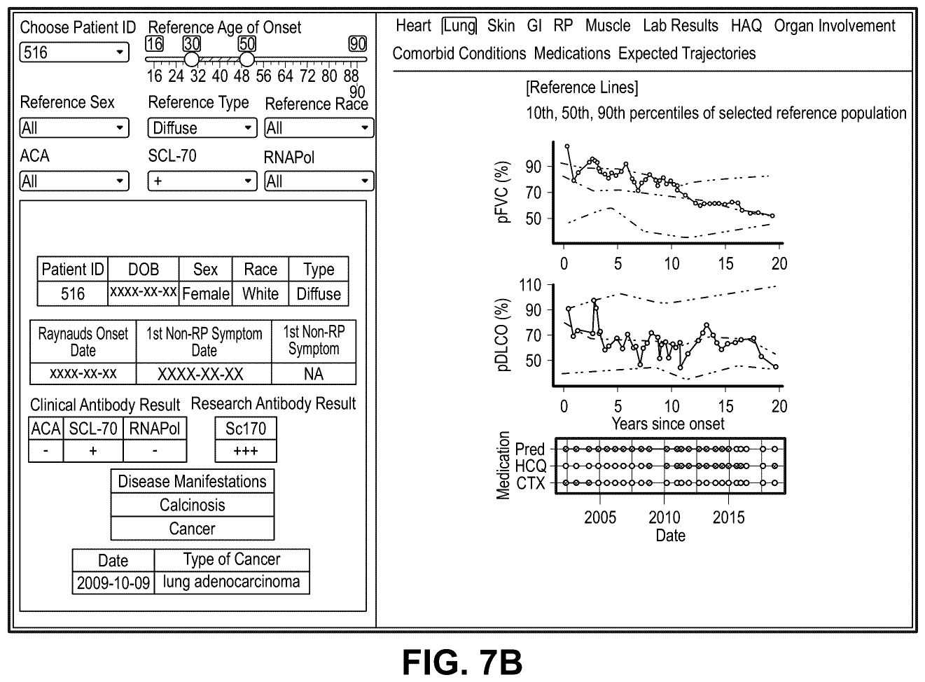

A utility of presenting an individual patient's data relative to those who share specific subgroup characteristics is demonstrated. In FIGS. 7a and 7b, a health trajectory of a white woman who developed diffuse scleroderma with anti-Scl-70 antibodies at 40 years of age is compared to other patients with similar demographic and clinical characteristics, providing insight into how individual and combinations of risk factors may modify a patient's likely health trajectory and outcome.

FIG. 7a shows lung trajectories of a patient and 10^{th}, 50^{th}, and 90^{th} percentile reference lines of an overall scleroderma population in one embodiment.

FIG. 7b shows lung trajectories of a same patient and 10^{th}, 50^{th}, and 90^{th} percentile reference lines of a selected subpopulation.

On a top panel (FIG. 7a), this patient's trajectory is compared with that of the overall scleroderma population. On the bottom panel (FIG. 7b), that same patient's trajectory is compared with a reference population that is similar to the patient (onset age of 30 to 50 years, diffuse type, Scl-70 antibody positive). These data illustrate how a reference population changes the interpretation and one's perspective. When comparing this patient to the overall scleroderma population, we observe that her pFVC trajectory declines from the 90^{th} percentile to 10^{th} over 20 years of follow up, dropping rapidly below the 50^{th} percentile line after 10 years. Relative to other similar patients, however, we see that her pFVC trajectory is better than that typically expected for the first 10 years of follow up and around the median afterwards.

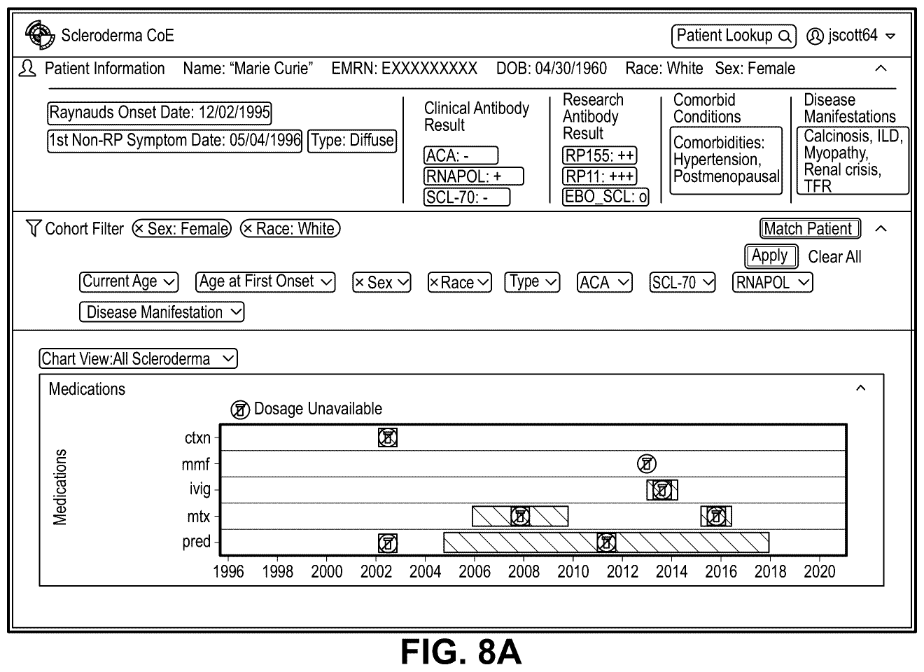

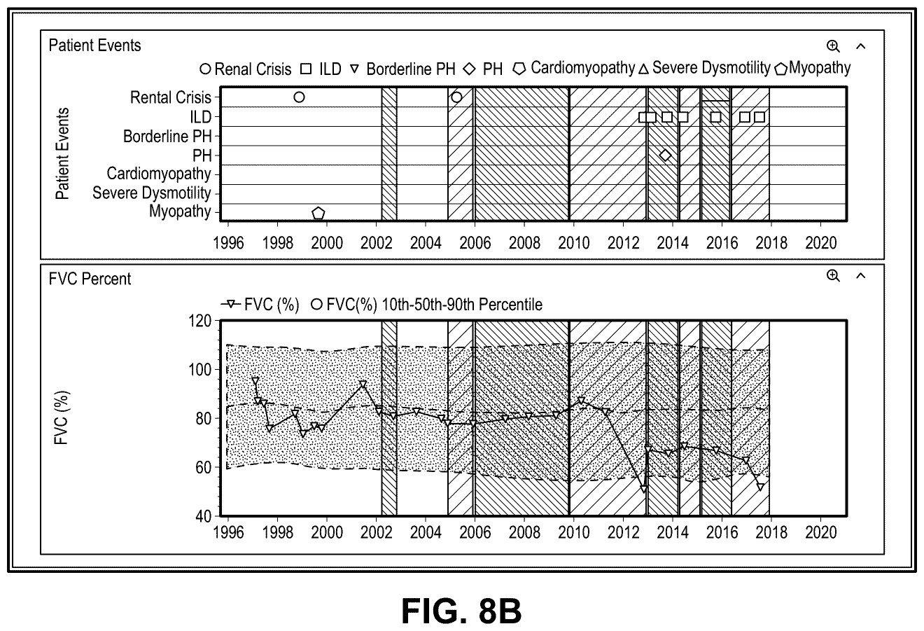

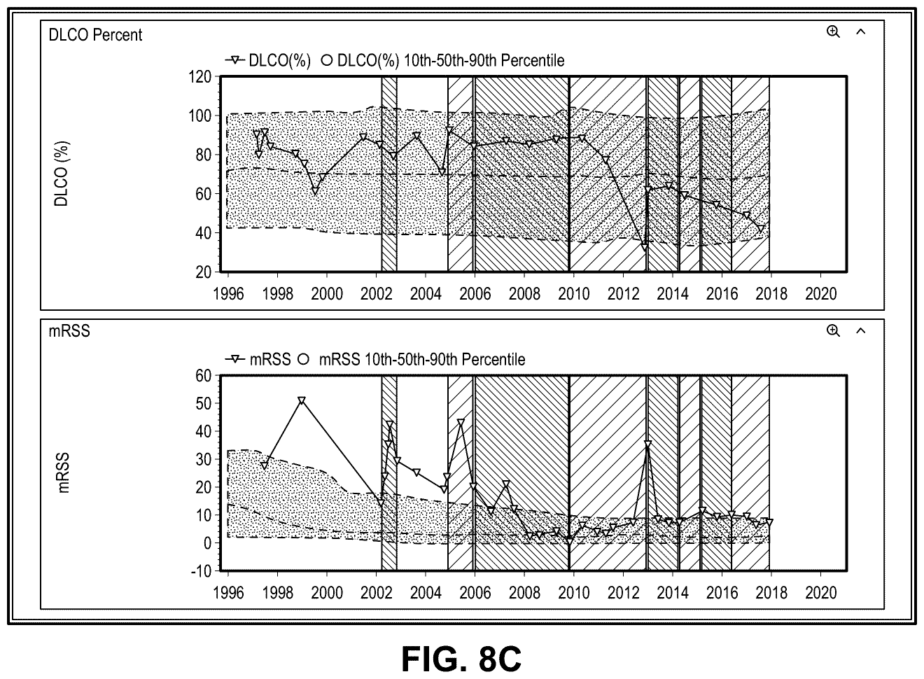

In FIGS. 8a-8c may be displayed as a single scrollable display by the VA tool in an embodiment. In FIGS. 8a-8c, users can select variables of interest to view only longitudinal data of the selected variables. Filtering of the reference population and the 10^{th}, 50^{th}, and 90^{th} percentile reference lines are also featured. In other embodiments additional or different filters may be used than as shown in FIG. 8a-8c. The VA tool is currently used by physicians in a clinic mainly for the purpose of effectively scanning through patient data to assess patients' past and current health status prior to patients' visits. By implementing this version within an EMR application, clinical studies discussed below now can be conducted. This was not possible using the VA tool alone.

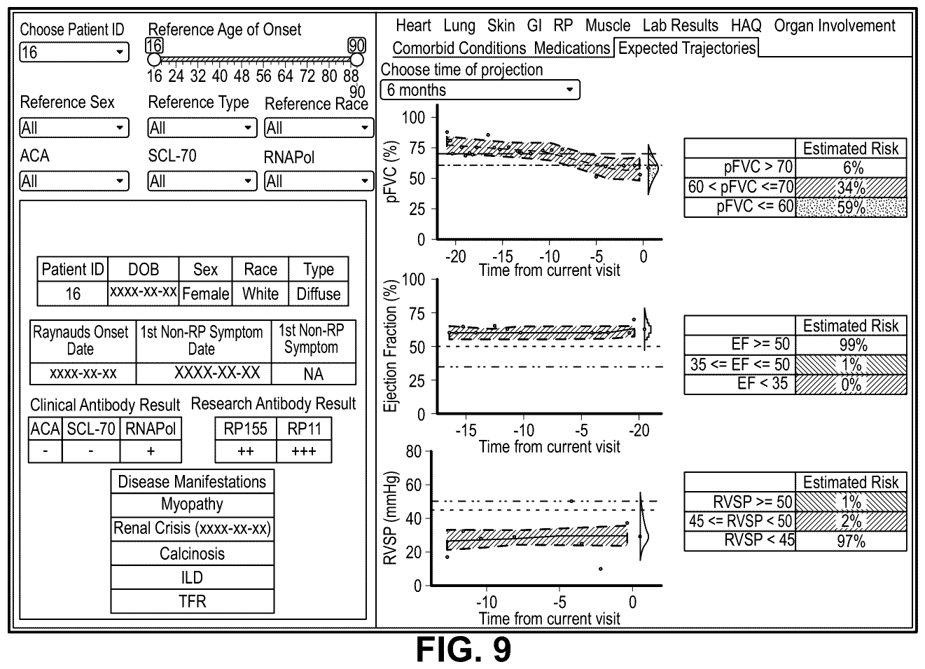

In addition to illustrating a patient's prior health trajectory, one goal is to improve risk estimation of a patient's likely future outcomes. In the VA tool, each patient's disease trajectory may be estimated and visualized with a 95% prediction interval for pFVC, EF, and RVSP using all available data (FIG. 9). Note that the predicted curves are obtained by jointly modeling multiple measures. The method is particularly useful when some measures have fewer data points observed compared to others. Patients generally have fewer observations for cardiac measures (EF, RVSP) and richer data for the pulmonary measures (pFVC, pDLCO). When there are only sparse data observed for a measure or a patient, we borrow strength from the other measures and from the entire cohort to produce more accurate and precise estimates.

Estimated risks of 6 critical events (3 outcomes each with 2 severity levels) in a next 6 months are displayed in FIG. 9. As the events are directly defined by the value of the longitudinal measures, we project an estimated health trajectory forward in time and calculate a risk of future events using an estimated uncertainty around the prediction. Areas of shaded regions indicate estimated risk of having each event. The estimated risks may be quantified and tabulated next to the graphs. This particular patient has a high risk of having a clinically significant restrictive ventilatory defect (93% for pFVC≤70%) and only a slight chance of having cardiomyopathy or PH (1% for LVE <50% and 3% for RVSP≥45 mmHg).

FIG. 9 illustrates an individual patient's trajectory in pFVC, EF and RVSP over time with an estimated risk of crossing high risk thresholds in these parameters within a next 6 months. A degree of uncertainty around these estimates is illustrated in the distribution of potential values projected forward at time 0 (the time of the current visit), and estimated risks of crossing certain thresholds is shown in tables on the right. In some embodiments, a time of projection can be changed by an end user to illustrate predicted estimates at, for example, 6, 12, 18 or 24 months in the future. In other embodiments, other times of projection may be used.

In complex, heterogenous diseases, personalized medicine strategies that harness and integrate knowledge from individual patients and populations have great potential to improve risk estimation and tailored decision making. Developing methods to bring new predictive models into clinical settings is highly important to demonstrate the value of these tools, foster the creation of a continuous learning health system, and enable future dissemination. The VA tool and statistical models allow flexible parameterization and can be applied to display clinical data and model health trajectories for other complex diseases. This framework provides a foundation that can be scaled and generalized for multiple clinical applications and disease states.

1. A method of providing an interactive patient-level data visualization and analysis tool that illustrates a patient's health trajectory across multiple organ systems, the method comprising:
integrating data from an electronic medical record system and one or more research databases into an analytics platform;
plotting, via a visualization tool, the patient's health trajectory; and
overlaying, by the visualization tool, data from an entire user-defined disease cohort as a reference group to visualize a disease course of the patient compared to courses of other patients, with a same disease, selected by a user.

2. The method of claim 1, wherein:
the data integrated from the electronic medical record system and the one or more research databases into the analytics platform is real-time-data, and
the real-time data is provided using FHIR technology.

3. (canceled)

4. The method of claim 1, further comprising:
performing calculations and processing of the data from the electronic medical record system and the one or more research databases for each patient individually and for all reference patients collectively.

5. The method of claim 4, wherein the performing of the calculations and the processing of the data from the electronic medical record system and the one or more research databases for each patient individually and for all reference patients collectively further comprises:
receiving new real-time data from the electronic medical record system and the one or more research databases; and
updating the calculations and the processing based on the received new real-time data.

6. The method of claim 1, further comprising:
receiving, by the analytics platform, filters to compare the patient's health trajectory to a user-specified subgroup based on demographic, clinical, and biological characteristics.

7. (canceled)

8. The method of claim 1, further comprising:
modeling, by the analytics platform, respective latent health states of disease patients based on using cardiopulmonary and cutaneous parameters.

9. The method of claim 8, further comprising:
projecting, by the analytics platform, the disease patient's health trajectory into a future;
calculating, by the analytics platform, respective probabilities that parameters of the disease patient will fall below or rise above clinically set boundaries; and
presenting the respective probabilities with a corresponding visualization of the parameters.

10. (canceled)

11. The method of claim 1, further comprising:
illustrating, by the visualization tool, data across multiple organ systems, the data including cardiac (left ventricular ejection fraction, right ventricular systolic pressure, and right heart catheterization data), pulmonary (percent predicted forced vital capacity and diffusing capacity), cutaneous (modified Rodnan skin score), gastrointestinal (Medsger GI severity scores and body mass index), peripheral vasculature (Medsger Raynaud's scores capturing damage including digital pits, ulcerations and gangrene), muscle (proximal muscle strength on a 0-5 scale), laboratory measurements, and patient reported outcomes (HAQ-DI).

12. A system for providing an interactive patient-level data visualization and analysis tool that illustrates a patient's health trajectory across multiple organ systems, the system comprising:
a processor; and
a memory connected with the processor, the memory including computer-readable instructions for the processor to perform a plurality of operations comprising:
integrating data tables from an electronic medical record system and one or more research databases into an analytics platform;
performing calculations and processing of the data from the electronic medical record system and the one or more research databases for each patient individually and for all reference patients collectively;
plotting, via a visualization tool, the patient's health trajectory; and
overlaying, by the visualization tool, data from an entire user-defined disease cohort as a reference group to visualize a disease course of the patient compared to courses of other patients, with a same disease, selected by a user.

13. The system of claim 12, wherein the data integrated from the electronic medical record system and the one or more research databases into the analytics platform is real-time-data.

14. The system of claim 13, wherein the real-time data is provided using FHIR technology.

15. (canceled)

16. The system of claim 12, wherein the performing of the calculations and the processing of the data from the electronic medical record system and the one or more research databases for each patient individually and for all reference patients collectively further comprises:
receiving new real-time data from the electronic medical record system and the one or more research databases; and
updating the calculations and the processing based on the received new real-time data.

17. The system of claim 12, wherein the plurality of operations further comprise:
receiving, by the analytics platform, filters to compare the patient's health trajectory to a user-specified subgroup based on demographic, clinical, and biological characteristics, wherein
the demographic, the clinical, and the biological characteristics include age at disease onset, race, sex, cutaneous subtype, and autoantibody status.

18. (canceled)

19. The system of claim 12, wherein the plurality of operations further comprise:
modeling, by the analytics platform, respective latent health states of disease patients based on using cardiopulmonary and cutaneous parameters.

20. The system of claim 19, wherein the plurality of operations further comprise:
projecting, by the analytics platform, the patient's health trajectory into a future;
calculating, by the analytics platform, respective probabilities that parameters of the patient will fall below or rise above clinically set boundaries; and
presenting the respective probabilities with a corresponding visualization of the parameters.

21. (canceled)

22. The system of claim 12, further comprising:
plotting, by the visualization tool, critical events of the patient, the critical events including clinically significant interstitial lung disease, severe interstitial lung disease, cardiomyopathy, pulmonary hypertension, mean pulmonary arterial pressure, severe gastrointestinal dysmotility, myopathy, renal crisis, and cancer diagnosis.

23. A non-transitory computer-readable medium having stored thereon instructions for a processor to perform a plurality of operations comprising:
integrating data tables from an electronic medical record system and one or more research databases into an analytics platform;
plotting, via a visualization tool, a patient's health trajectory; and
overlaying, by the visualization tool, data from an entire user-defined disease cohort as a reference group to visualize a disease course of the patient compared to courses of other patients, with a same disease, selected by a user, wherein
the data integrated from the electronic medical record system and the one or more research databases into the analytics platform is real-time-data.

24-25. (canceled)

26. The non-transitory computer-readable medium of claim 23, wherein the plurality of operations further comprise:
performing calculations and processing of the data from the electronic medical record system and the one or more research databases for each patient individually and for all reference patients collectively, wherein
the performing of the calculations and the processing of the data from the electronic medical record system and the one or more research databases for each patient individually and for all reference patients collectively further comprises:
receiving new real-time data from the electronic medical record system and the one or more research databases; and
updating the calculations and the processing based on the received new real-time data.

27-29. (canceled)

30. The non-transitory computer-readable medium of claim 23, wherein the plurality of operations further comprise:
modeling, by the analytics platform, respective latent health states of disease patients based on using cardiopulmonary and cutaneous parameters.

31. The non-transitory computer-readable medium of claim 30, wherein the plurality of operations further comprise:
projecting, by the analytics platform, the patient's health trajectory into a future;
calculating, by the analytics platform, respective probabilities that parameters of the patient will fall below or rise above clinically set boundaries; and
presenting the respective probabilities with a corresponding visualization of the parameters.

32-33. (canceled)
